(19) United States
(12) Patent Application Publication
(10) Pub. No.: US 2004/0110193 A1
(43) Pub. Date: Jun. 10, 2004

(54) Methods for classification of biological data

(75) Inventors: Arthur L. Castle, Gaithersburg, MD (US); Michael R. Elashoff, Gaithersburg, MD (US); Kory R. Johnson, Gaithersburg, MD (US); Mark W. Porter, Gaithersburg, MD (US)

Correspondence Address: MORGAN LEWIS & BOCKIUS LLP, 1111 PENNSYLVANIA AVENUE NW, WASHINGTON, DC, 20004, US

(73) Assignee: Gene Logic, Inc.

(21) Appl. No.: 10/633,306

(22) Filed: Aug. 4, 2003

Related U.S. Application Data
(63) Continuation-in-part of application No. 09/917,800, filed on Jul. 31, 2001.
(60) Provisional application No. 60/400,099, filed on Aug. 2, 2002.

(51) Int. Cl. C12Q 1/68

(52) U.S. Cl. 435/6

(57) ABSTRACT

Methods for classification of biological data including the use of mathematically based classification models are disclosed. In preferred embodiments, gene expression data derived from biological studies is used to classify events. The methods use a version of a linear discriminate model for deriving markers according to a technique whereby the model is limited to scoring the predictive ability of genes without making assumptions about inter-gene relationships, relative magnitudes or redundant information. In an associated method for building a fully informative database of biological information, biological studies are performed on a continual basis and linear discriminate models are used to provide rapid and efficient updates to gene markers.

### CONTINUING APPLICATION INFORMATION

This application claims priority to U.S. Provisional Application No. 60/400,099, filed Aug. 2, 2002, and is a continuation-in-part of U.S. application Ser. No. 09/917,800, filed Jul. 31, 2001, the disclosures of which are incorporated herein by reference in their entirety.

### BACKGROUND OF THE INVENTION

Methodologies for making statistical inferences about a particular pathology or state at the cellular or sub-cellular level based upon the activity of genes, such as whether or not a particular cell is, or is not a tumor cell, are known. In particular, statistically-based methodologies are used for identifying characteristic genes or gene sequences which can differentiate between different physiological states. Hence, these methodologies seek to identify a group of “gene markers” for a particular condition. These tentatively predictive gene markers are often times found by first observing which genes pass some arbitrary threshold, such as two-fold increase in expression level in a large screen using a single treatment. For example, a single or relatively few samples are run on a large parallel measurement device such as a microarray or differential display. All genes in this system are then ranked by the fold change. In some cases an additional p-value cutoff is used to reduce the number of genes.

It has been found that these systems are inherently flawed and likely to give unreliable results for several reasons. By establishing a ranking based on magnitude of response, one has implicitly assumed that all genes belong to the same population when, in reality, they often do not. Different genes have different response ranges. For example, the expression level for a heat shock protein gene may change 100-fold due to strong physiological stress, while the expression level for another gene may only change 1.4 fold. These same genes may respond to a weak stress that causes the expression level of a heat shock protein gene to change between 20 to 60-fold while almost no change is seen in the expression level for the second gene. The degree to which expression of a gene changes must be considered within the context of its normal dynamic range and its tendency to vary randomly within that range.

Statistical tests in use today tend to make certain underlying assumptions concerning the parameters defining the populations from which the samples are collected. One example is the t-test. While using the t-test when replicates are available may reduce some of the problems associated with drawing inferences based on, e.g., fold change, the t-statistic may not always be reliable when only a small number of replicates are available, or when the variance between samples is significantly different. This is because the reliability of a t-distribution to infer significance depends on how well variance has been estimated and whether the variance between the samples, or groups of samples, is similar.

An additional problem occurs when an observed change is assumed to be due solely to the physiological stress applied. A classical correlation does not necessary mean causation. A good marker must not solely discriminate between the control and experimental condition within the context of the experiment. It must also not be affected by variables not captured within the experiment. Referring to the example above, only mild changes in heat stress could greatly affect the fold change of the heat shock gene. An experiment that looks only at fold changes based on a small, controlled study, however, cannot possibly capture this confounding condition.

While using more complex multivariate modeling methods, such as rule based methods, can mitigate some of these problems, they may introduce other problems. For example, one may use both a statistical and rule based method to select marker genes as follows. One may observe that 10 genes seem to be affected by an outside stimulus according to a statistical test and of these 10 genes, 8 are required to meet a certain criterion before they are accepted under the rule-based method. This type of model implies an association between the 10 genes and the end affect of the stimulus, as defined by the rule. While one may eliminate the previous problem where the heat shock gene is affected by things not captured in the experiment, we introduce a new problem: the rapidly decreasing likelihood that all of these genes are associated as the number of genes are increased, and/or restrictions are placed on the nature in which the measurements need to be combined. Even though we observed that all 10 genes changed in the experiment, one needs to determine if the rules used to evaluate the data “over-fit” the data. The nature of the typically restrictive rules characterizing rule-based methods can extend to assuming that changes in multiple genes are a result of the same outside stimulus, or that each gene provides a measure of an independent aspect of the final prediction. For example, if two genes have the same profile across all samples, one may conclude that the genes represent redundant measures of the same effect, or that each gene can correctly pick different samples well and thus can serve as different partial markers.

Cross validation may be used to assess the accuracy of the predictive model. In one approach, cross validation includes the removal of some samples in the training set, using the remaining samples to select genes according to the method, and then testing the new set of genes against the removed samples to estimate the fit of the rules or formula underlying the method for combining gene measurements to make predictions. This process needs to be repeated many times so that a confidence interval can be placed around the rules or formula. Re-sampling techniques, such as jacknife or bootstrap, may be used to assess biases, means and standard deviations of statistical parameters. These types of methods for evaluating computed statistics are, however, very computer intensive and rely upon an abundance of available samples.

The more restrictive the rules for combining genes the less likely each round of cross validation will match the initial observations. For very restrictive rules such as neural networks, one may need to perform thousand of rounds of cross validation whenever genes are added or removed. However, there must be enough observations available so that a unique selection of a samples is available for cross-validation whenever the model is updated. This poses problems for many of the more restrictive methods employed today. If unique samples are not available to validate the model, then the usefulness in using a model that requires extensive cross-validation whenever the model is updated diminishes.

### SUMMARY IF THE INVENTION

The above needs are met, and the shortcomings of prior art are overcome by the methods of the invention. The invention provides a method of classifying biological data that includes a linear discriminate analysis (LDA) model for classification of samples and the identification of marker genes. This method may be used to identify marker genes associated with toxicity classes in general, toxicity to a particular organ (e.g., the liver), pathologies of a particular organ (e.g., hepatitis, steatosis) or particular toxic compounds. Methods are also provided for classifying data through a process of iteratively using and updating LDA models in parallel with performing biological studies for purpose of building a fully informative database of classifications. Such a database may be used to assist with, among other things, obtaining information relevant for drug screening and pathway discovery and/or generally explaining or predicting mechanisms of action for biological processes such as toxicity.

The invention also includes a method of identifying one or more marker genes whose level of expression predicts a biological response in a cell or tissue. This method comprises the steps of obtaining gene expression data for at least a test cell or tissue group and a control cell or tissue group, in which each of the test and control groups comprises a collection of cell or tissue samples, and analyzing the gene expression data to identify one or more marker genes by a linear discriminate analysis that does not take into account the factors of the magnitude of the difference in gene expression for each gene between the test and control groups and the behavior or identity of other genes detected in the samples or across samples, or combinations thereof, thereby identifying one or more marker genes whose level of expression predicts a biological response in a cell or tissue.

The invention further includes a method of building a classification database, comprising the steps of obtaining gene expression data for at least one test group of cell or tissue samples and at least one control group of cell or tissue samples; identifying marker genes by a linear discriminate analysis capable of discriminating between the test and control groups and storing this information in the database; evaluating the predictive ability of at least one of the markers; and refining the database with information derived from additional biological studies.

### BRIEF DESCRIPTION OF THE DRAWINGS

The accompanying drawings, which are included to provide a further understanding of the invention, are incorporated in and constitute a part of this specification, illustrate preferred embodiments of the invention and together with the description serve to explain the principles of the invention. In the drawings:

FIG. 1 is a plot showing results from a cross-validation study for a preferred hepatoxicity LDA model incorporating the principles of invention. Specifically, the plot shows that the model produces very similar true positive and true negative rates when comparing a model based on full training set samples and a model based on 1/3 random drops in the training set.

FIG. 2 is a plot showing the true positive rates for pairs of hepatoxicity models that include a group of several known toxic compounds. For one member of each pair, the true positive rate is calculated using a particular group of compounds (included), while for the other member of each pair, the true positive rate is calculated with the omission of data related to one compound in the group (not included). More specifically, the plot shows the true positive rates for identifying a sample as belonging to a toxin-exposed group when a particular compound is and is not included in the training set for the LDA model.

FIG. 3 is a flow diagram illustrating a preferred method for building a fully informative database using LDA models in accordance with the principles of invention.

FIG. 4 is a schematic illustrating a preferred toxicology classification database developed using the methodology of FIG. 3.

### DETAILED DESCRIPTION OF THE INVENTION

The LDA model and method for using the LDA model of the invention is geared towards identifying markers for predicting a particular category of physiological change, but with the two advantages of a robustness in the model that allows for efficient updates to models and a minimization in the amount of assumptions about inter-gene relationships needed to classify data. As to the first advantage, frequently changing needs for classification of data are better served by a model that can be readily updated. In this aspect of the invention, markers selected using the LDA model of the invention greatly reduce undesirable over-fitting of the model to the training samples. In a preferred embodiment, a linear discriminate model is used that is based upon a spherical discriminate model, as described in S-PLUS 2000 Guide to Statistics, Vols. 1 & 2, Data Analysis Products Division, MathSoft, Seattle, Wash. (May 1999), which is herein incorporated by reference in its entirety.

As to the second advantage, the ongoing efforts to understand complex biological processes based on classification models are better served by a model that tends to minimize a priori assumptions about the biological data on which it is based. This is, in particular, because the analysis of classification data tends to be less burdened by biases resulting from assumptions made about the data, which can limit the usefulness of the model (especially if the model is frequently evaluated and/or updated in response to, or for purposes of, identifying parameters for new biological studies addressing research needs). Measuring true variability across as large and diverse a population as possible and using a linear discriminate metric, as opposed to a magnitude or significance of change metric, mitigates the difficulties, biases and/or unjustified assumptions associated with the interpretation of magnitude, variability and confounding factors when selecting biological responses as markers for biological activity. By using an LDA model, biological responses are ranked solely by their ability to distinguish one sample group from another, without the need for enforcing many of the a priori assumptions about an underlying biological process that is common to many of the methods in use today. This is advantageous because assumptions on magnitude, variability across samples or interdependencies between, e.g., gene expression, in the known classification models are typically not verifiable. Biological systems are very complex and many non-mutually exclusive relationships exist between the regulation of genes and a particular biological condition, e.g., toxicity. Even if one were provided with a database that captured all of the true inter-gene regulation, an accurate modeling of the complexities of inter-gene relationships would require many more observations of gene regulation than would be relevant to the gene regulation underlying the biological classification of interest.

In another aspect of the invention, the LDA model interprets and weighs each gene based on its ability to discriminate, without incorporating estimates of inter-gene relationships with, e.g., a correlation matrix. This allows a cross-validation to more closely resemble the model's fit to the training set, and without over-fitting.

According to one embodiment of the invention, a method for classifying a biological sample (based on the expression level of a gene or genes in the sample) as a member of a first group, such as a toxin-exposed group, or a second group, such as a control group or toxin-unexposed group, where the groups include samples of gene expression representative of a training set, includes the steps of computing parameters associated with first and second sets of probability distributions for each group, the probability distributions and parameters for the expression level of each gene being derived independently of any other gene in the training set, and selecting a set of markers from the genes. This marker selection step includes the steps of computing a discriminative ability score for each observation of gene expression for each gene in the first group according to a scoring function, computing a discriminative score for each gene in the first group including counting the number of times that the discriminate ability score is greater than a first predetermined number for each gene in the first group, and then selecting a set of marker genes including the step of selecting those genes whose discriminative score is above a second predetermined number. Once the markers are selected, the method then classifies the sample as a member of one of the first and second groups based upon a probability that the sample is a member of the first group. The discriminative score may also be computed from the number of times that a gene expression level is both greater than the first predetermined number in the first group, and less than this number in the second group.

In another aspect of the invention, a method for classification uses an LDA model that selects from a database a diversity of samples covering a wide variety of controls, and a wide variety of genes for the model without making assumptions about mutual information contained in the genes. In this method, a good fit is achieved on a consistent basis. This is advantageous because, whenever assumptions about mutual information are incorporated into a predictive model in order to improve predictive quality, many more observations than variables are needed, as the estimation involves n squared parameters (both the correlation of all the genes and their individual distributions) rather than n parameters.

In another aspect of the invention, an LDA model that is limited to selecting markers that simply distinguish one sample from another permits the building of a fully informative biological database where new data is constantly added and classification models are continuously updated and revised in an efficient and streamlined manner. If an extensive cross-validation, such as a monte-carlo or other stochastic method of cross-validation, is needed every time new information is incorporated into the model (i.e., new genes are added or others are removed), updates to the model will require extensive computer resources. This can result in extensive lead times before an updated model is available. This is particular true when using non-linear predictive models, such as a neural network with hidden layers. According to this aspect of the invention, extended delays associated with cross-validation and interpretation of model results are minimized.

According to another aspect of the invention, a method for building an optimally informative classification database is provided. Biological studies are designed to capture events such as toxicity by exposing multiple animals or cell or tissue cultures to a stimulus such as a toxin at multiple doses with appropriate negative controls. The parameters of the studies are initially determined by perceived need based on historical literature and dose ranging experiments where classical parameters are measured, e.g., histology, clinical chemistry such as ALT and AST, and hematology. This perceived need is only an initial estimate of the samples that are needed to build an optimally informative database. Accordingly, it is necessary that the classification database is sufficiently robust to allow rapid modification pursuant to continuous feedback as to which prior perceived needs have been met by previously conducted studies, where more studies and classification protocols need to be developed, and whether a hypothesis generated from perceived needs is reasonable.

According to another embodiment of the invention, a method for building a biological database for classification includes the steps of providing an LDA model and performing the steps of (a) running tentative test and control studies according to perceived needs for biological data that can be used to identify markers for an interesting physiological change; (b) deriving tentative test and control groups from the studies; (c) deriving tentative markers using the LDA model; (d) evaluating the predictive ability of the markers by applying markers to samples known to be members of the group; and repeating steps (a) through (d) until the markers return a true positive and false positive rate that meets a predetermined threshold acceptance level. This method may be used for deriving a library of classification models for toxicology classification, such as a generally toxicology classification model, a classification model for pathologies, or specific types of compounds.

The method of building and updating a classification database is preferably sufficiently robust as to incorporate the results from up to several studies per week. Each of these studies may frequently exceed over a million data points, e.g., a million measurements of gene expression value for each of a plurality of samples derived from the study. Meta-data is also collected from the studies, including study characteristics, histopathology, clinical chemistry, hematology and other assays.

The particular usefulness for classification of biological data obtained using the methods of the invention, whatever the end purpose, includes any known use for the classified data and the classification criteria. Accordingly, whether the classified data obtained using the methods of the invention are used for interpreting the biological effects of a candidate drug treatment, or for some other purpose, the scope of the invention is not limited by that purpose.

Additional features and advantages of the invention are set forth in the description that follows. The features and advantages of the invention are realized and attained by the methods particularly pointed out in the written description and claims below, as well as in the appended drawings. It is to be understood that both the foregoing description and the following more detailed description are exemplary and explanatory and are intended to provide further explanation without limiting the scope of the invention as claimed.

Linear Discriminate Analysis (LDA)

“Biological studies” refers to any methodology for measuring a physiological change in one or more cells in response to an outside stimulus applied either directly to the cell or as an indirect by-product of some other stimulus, either in vitro or in vivo, from which gene or protein expression, or another type of biological response may be derived. This term also encompasses methods of measuring a physiological difference in a sample compared to another sample, such as a disease state, etc. This term, therefore, also encompasses studies to stratify patient samples or populations, for instance, to predict a response to a drug or to select specific patient populations, such as for a drug trial. Examples of such biological studies are disclosed in International Publication Nos. WO 02/10453, published on Feb. 7, 2002; WO 01/25473, published on Apr. 12, 2001; and WO 99/64626, published on Dec. 16, 1999, all of which are incorporated herein by reference in their entirety.

Biological samples” or “samples” refers to any processed data from a biological study that has been digitized into a set of points, or numbers, describing the particular biological state (whether known or unknown), which may be analyzed according to mathematical methods defined over a space containing those points. Examples of samples may include gene expression samples, but may also include protein, metabolites, or any other mass biological or chemical measure samples.

“Meta-Data” refers to information about the biological study or studies that produced the biological data, other than the samples derived from the experiment, which may be informative about a particular group or classification suitable for the samples derived from the experiment, and/or the reliability of the samples as data points for classification. Meta-data may also include information about the time length between administering a stimulus and measurement of a biological response contained in samples, the method of administering the outside stimulus, the dosage level, or any other relevant chemical or biological assay. For example, meta-data content may include study characteristics relating to histopathology, clinical chemistry, hematology and other assays, e.g., weight of an organ or animal, a description of behavior of an animal, an ECG or any kind of diagnostic test.

“Physiological change” refers to any physiological change in an organism that may be detected, such as a change used to uncover drug pathways for treatment of a pathology and/or identification of mechanisms of action related to a particular cellular state or pathology, either directly or indirectly as a result of the observed physiological change.

“Physiological characteristic” refers to any characteristic in an organism or cell or tissue sample that distinguishes that organism or cell or tissue sample from another organism or cell or tissue sample. Such characteristics include, but are not limited to, metabolic state, presence of uncontrolled growth (cancer or neoplasia), etc.

“Gene expression data” refers to data concerning the suppression, induction or regulation (up-regulation or down-regulation) of one or more genes that may be obtained through any known method. Such data may be quantitative.

“Marker” or “markers” refers to biological responses that are selected using a classification math model for purposes of distinguishing, or discriminating between, a sample (or samples) relating to a physiological change and sample(s) that do not relate to the physiological change. An example of “markers” is a collection of genes, with each gene as an individual marker.

“Test group(s)” refers to a group of samples collected from biological studies that represent a physiological change that is classifiable as a particular type of biological response. Test groups may be derived either through a supervised grouping protocol (e.g., similarities in meta-data for samples), an unsupervised grouping technique (e.g., a group-selecting mathematical algorithm) or a combination of the two. In the preferred embodiments, groups are assembled using a supervised technique including a review of the meta-data associated with the biological studies. Examples of classifications embodied in test groups may include, but are not limited to, general toxicity, specific types of toxicity such as a particular type of pathology, and/or toxicity associated with a particular compound or compounds.

“Control group” refers to a group of samples collected from biological studies that represent a normal physiological state or healthy condition (i.e., no outside stimuli), or a group of samples from biological studies that reflects a physiological change resulting from an outside stimulus including one or more of a vehicle, pharmacologic dose, negative control or other varieties of outside stimuli resulting in a physiological response that is not of interest in classification but which is used to assist with identifying markers that predict only the true cause of physiological change. A training set for a classification model includes a control group and a test group, either collectively or individually.

A “fully informative classification database” is a classification database that seeks to capture all samples relevant to fulfilling actual needs for understanding one or more physiological changes. Actual need refers to the need for samples from studies which, when examined using classification techniques, reveal the true underlying biological process. In contrast, a perceived or hypothesized need is only an estimate of the type of samples that are needed to fully understand an underlying biological process. Perceived needs are a starting point in building a fully informative database because studies and examination of classification metrics based on these studies are necessary before a more informed decision can be made on the type of studies that are needed to meet actual needs. The collecting of studies for the fully informative database includes obtaining samples which encompass controls that could conceivably effect, either directly or indirectly, a particular pathology so that a true underlying biological process can be isolated using classification techniques.

“LDA” refers to any linear statistical model that derives markers for discriminating between samples that relates to a physiological change and that does not relate to the physiological change based upon a predetermined and often times tentative (i.e., subject to change) control and test groups. As used herein, an LDA model includes any linear method that minimizes or eliminates significant a priori assumptions on variance or co-variance, relative magnitude or similarity in response across a group of biological data points, e.g., digitized gene expression levels, found in the samples. An LDA model measures the discriminative ability of each biological data point on an individualized basis, irrespective of the behavior of other biological data points, such as relative magnitude, variance, redundancy in information, etc. As used herein, an LDA model refers to any mathematical technique that embodies the above definition and is not limited to the embodiments disclosed herein.

An LDA model is well suited for meeting the needs of an in-development, fully informative classification database where biological information affecting the validity of the model is received in large quantities and on a continual basis. This type of database is found to be difficult to implement effectively if other, less robust classification methods are employed because training sets and hypothesized needs for biological studies are under constant revision. An LDA model meets these demands because the model may be implemented so as to minimize extensive cross-validation of markers. Additionally, the model's results are relatively easy to interpret, and the model is able to predict a sample's group membership with accuracy.

Other less robust, or static models, e.g., neural networks, typically require extensive cross-validation because they tend to over-fit a training set. When a model tends to over-fit a training set, it correspondingly tends to provide poor predictive ability for samples that are not part of a training set. Some of these static models are even less reliable as a predictive metric because the assumptions underlying the prediction assume many unverifiable relationships in the data, either because there are either insufficient samples to verify the assumption, the true biological processes are generally too complicated to derive using an analytical model, or the verification for the assumption can only be found through an unreasonable number of examinations of unrelated biological processes. As such, predictions based upon these models typically have a limited usefulness when used as a general classification tool and in particular, when these models are used in the context of frequently changing assumptions underlying the model parameters and changes in the training set.

In a preferred embodiment, the training set for an LDA model draws gene expression samples from Gene Logic's ToxExpress® database. In this embodiment, samples include both a large number of genes and a large set of samples for the training set. For example, in a general hepatotoxicity classification model, at least several hundred control samples and several hundred test or experimental samples are included in the training set. Results are improved by including more samples in each group, and several thousand samples in each group may be used. Similarly sized sample groups may be used in predictive pathology models, such as the hepatitis model.

Samples in a training set may be sorted into groups using meta-data or published information related to the samples, or based on the judgment of the scientist assembling the training set. Analytical methods such as PCA logistic regression may also be applied to data points, but the criteria for sorting rely on being confident that a real effect was observed.

Within any sample contained in the training set, all genes are evaluated for their predictive ability. Thus, no a priori assumptions are used to reduce the number of candidate markers. This results in sometimes over 26,000 gene expression levels being evaluated for any given sample.

In a preferred embodiment, an LDA model is used to identify gene markers using a first and second tentative group of samples for toxin classification. The first and second groups refer, respectively, to samples grouped into a test group (toxin-exposed, or samples exhibiting a toxic effect) and control group (non-toxin-exposed, or samples exhibiting no toxic effect). The method is based on a discriminate score for each individual gene in each sample, regardless of the behavior of other genes in the sample or across samples, the relative magnitudes of response, or perceived redundancies in gene response. In other words, each gene is given equal weight as a predictive marker. A scoring function is used that is preferably based on an assumed normal distribution for the first group and for the second group. The discriminate score for the gene is determined by summing the instances where the scoring function returns a value for each sample that is greater than a predetermined significance level. An example from the preferred embodiment follows.

Let Xᵢ be the i^{th} expression level in group 1, Xᵢ, i=1 . . . t, for a gene, and Yⱼ be the j^{th} expression level in group 2, Yⱼ, j=1 . . . n, for the gene. Each of the samples are assumed to be taken from respective normal distributions, X˜n(m1,v1) and Y˜n(m2,v2) where m1, m2, v1 and v2 are estimators of the true mean and variance for each distribution based upon the respective set of t expression levels Xᵢ and n expression levels Yⱼ. The formulas that may be used for computing the estimators (m1, v1) and (m2, v2) are well known and need no further explanation here.

Define a scoring function ƒ₁(z) as follows:

$${f}_{1} ( z ) = \frac{( \frac{1}{\sqrt{v1}} ) {ⅇ}^{- \frac{1}{2} {( {z}_{i} - m1 )}^{\frac{2}{v1}}}}{( ( \frac{1}{\sqrt{v1}} ) {ⅇ}^{- \frac{1}{2} {( {z}_{i} - m1 )}^{\frac{2}{v1}}} + ( \frac{1}{\sqrt{v2}} ) {ⅇ}^{- \frac{1}{2} {( {z}_{i} - m2 )}^{\frac{2}{v2}}} )}$$

The score for a gene as a measure of its ability to discriminate between group 1 and group 2 can be determined by counting the number of times, P₁, that ƒ₁(Xᵢ)>½ for i=1 . . . t and the number of times, P₂, that ƒ₁(Yⱼ)<½ for j=1 . . . n. ƒ₁(Xᵢ) is the discriminate ability (a component of the summed discriminate score) for the i^{th} expression level of gene X in group 1, while ƒ₁(Yⱼ) is the discriminate ability for the j^{th} expression level of gene Y in group 2. X and Y refer to the same gene in two different sample sets.

A significance level of, e.g., ¾ and ¼, respectively, for ƒ₁(Xᵢ) and ƒ₁(Yⱼ) may be used in place of ½ if a more conservative scoring metric is desired. The discriminate score, P, for the gene is then (P₁+P₂)/(n+t), which will vary between 0 and 1.

By comparing the score P, to scores obtained for other genes, a set of marker genes is derived, e.g., by selecting only those genes having a cumulative discriminate score greater than some number S. Depending on the number of samples, S may have the value of about 0.6, 0.7 or even higher. If only a relatively small number of samples are available for groups 1 and 2, e.g., if group 1 relates to samples from studies for a particular compound, as opposed to samples collected from a group of compound studies, it may be preferable to assign a higher number to S, as the number of gene markers that can be reliably used as predictive markers is typically lower, given the training set.

In other embodiments, weightings may be applied to some of the samples in groups 1 and 2 for purposes of discounting, or enhancing the influence that one or more samples will have on the selection of markers. This may be desirable if, e.g., one or more markers are believed to contain noise or one or more markers are believed to be more representative of the group for which markers are desired.

Cross-validation

Selected markers may be cross-validated to confirm that the markers are good discriminators for a given characteristic, e.g., pathology. This may include verification that there is no over-fit to the training set, or that the marker selection was not overly biased by a particular type of sample in the training set, e.g., a particular compound type. For example, a cross-validation protocol may compute a first and second set of markers where the first set corresponds to a full training set and a second set corresponds to the first training set with one or more samples removed. Under this protocol, the markers derived from the full training set would be validated if the same, or nearly the same markers are selected from the second training set. In a preferred embodiment, a cross-validation scheme tests the validity of a set of markers by dropping ⅓ of the samples from the training set and then re-computing the markers to see whether the same markers are selected. More specifically, markers derived from 8-10 random selections of training sets having ⅓ sample drops are compared with markers derived from the full training set. Other validation methods may also be performed to ensure that the expression pattern driving the selection of markers was not due to a delivery medium or other type of control that may have affected the outcome of a study. The markers may be tested against samples derived from studies where an organism is exposed to negative controls, i.e., as a drug or substance thought to cause a change in gene expression but not resulting in a measurable physiological change, such as in toxicity studies. An example of this effect is found in the liver. Because the liver metabolizes most foreign chemicals entering the body, the expression level of a gene may change due to adaptive rather than maladaptive effects. Therefore, it is desirable to examine these negative controls, along with low dosage samples, to help distinguish non-toxic adaptive gene expression responses to external stimuli from the real indicators of toxicity.

As compared to other methods for classification using statistical models, rule-based models, or combinations of both, it has been found that the LDA model of the invention requires minimal cross-validation when a model is updated. In particular, it has been found that when the models are based on a sufficiently large training set, a relatively small number of random variations in the training set, e.g., 8-10 random variations, are sufficient to cross-validate the model. As mentioned above, this relatively quick and efficient validation protocol is well suited to methods of high-speed selection of markers, which are needed when study goals or classification interests change, such as when building a fully informative classification database.

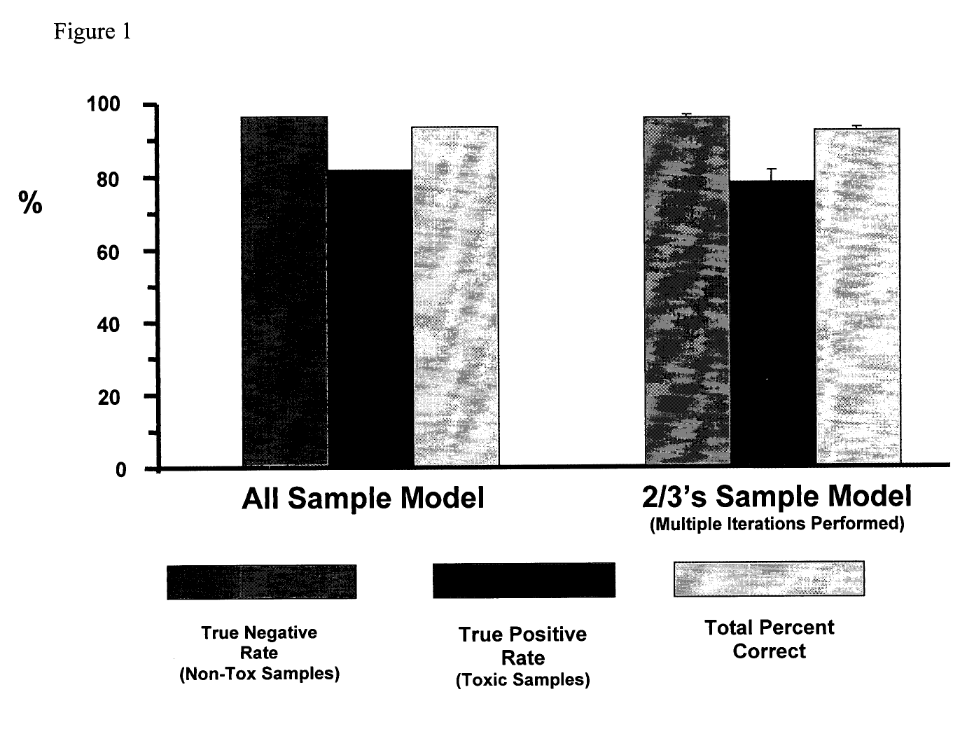

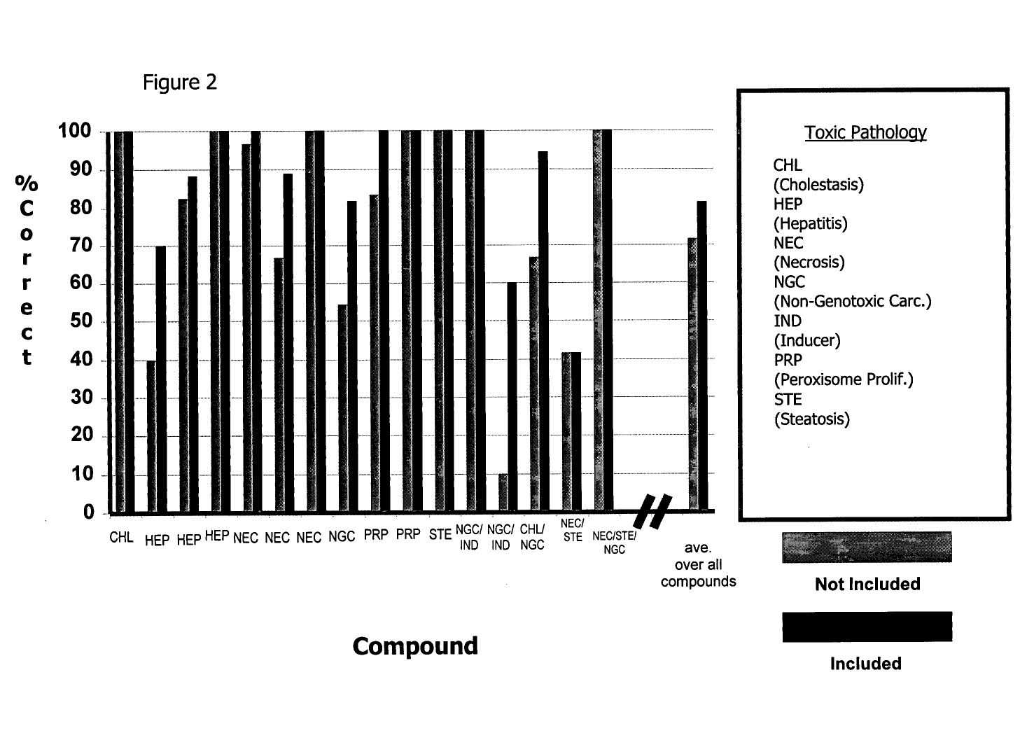

Examples of results from a cross-validation study using the LDA model of the preferred embodiment for classification of a general hepatotoxicity discriminate model is provided in FIGS. 1 and 2. In FIG. 1, a comparison between the true positive, true negative and total percent correct are shown for the model including a full training set (“All Sample Model”) with a model derived from a random ⅓ drop in the training set (“⅔'s Sample Model”). Specifically, in the ⅔'s Sample Model, a random sample of two-thirds of the samples were selected to create a training set. The remaining ⅓ of the training set was composed of genes that were predicted to be “tox” for the tox sample group, or “non-tox” for the non-tox sample group. In one example of cross-validation, eight such training sets were prepared in this manner, and two genes were picked that were present in the ⅔ portion of each training set. Their classification was then determined as to whether or not their expression levels were considered to change upon exposure to a toxin, and the results were compared to the results using full training sets.

The percentage correct rates shown for the model based on all training samples (% correct) vs. the model with only ⅔ of the training samples are very similar. If fewer samples are available for a training set, the results will vary more, as one would expect. In FIG. 2, a plot of true positive rates for the general hepatotoxicity model for compounds that are included and not included is shown. This plot shows the stability of the model and its ability to predict toxicity when any one type of compound is used to build the model, as is evident from the average true positive rates shown.

In a preferred embodiment, it has been found that when an updated general hepatoxicity model exhibits a true positive rate greater than 80% and a false positive rate less than 5%, when the markers are tested against samples not in the training set, the markers selected by the model are sufficient for predicting membership in the group insofar as there is little concern that the markers are over-trained by the training set.

The above ⅓ random sample drop cross-validation protocol and 80% true positive/5% false positive validation metric for determining whether there is an over-fit to the training set require a sufficient number of unique samples available for a training set. If only a relatively small number of samples are available for the training set, and/or if the samples are mostly homogeneous in nature, i.e., they are taken from similar biological cohorts in the studies, meaning measurements taken at the same time, same dosage level, same drug, etc., then the model will usually be over-fit to the training set. But, when a sufficient number of heterogeneous samples, i.e., samples taken from a wide variety of biological cohorts, can be accumulated, such as in the case of a general toxicity model, the aforementioned cross-validation scheme using random ⅓ random sample drops with a 80% true and 5% false positive validation error rate may reliably verify that the model is not over-fit to the training set and thus no exhaustive cross-validation protocol is needed every time that the model is updated.

Probability of Group Membership

When using an LDA model of the invention to predict membership in group 1, the scoring function ƒ₁(˜) may be applied to a new sample Z and a resulting probability of membership in group 1 may then be calculated. Other methods of computing the probability of group membership may be used. In a preferred embodiment, a ceiling and floor are enforced on the data so that an expression level Zₖ in any one of the m marker genes for sample Z does not cause the quantity

$$( \frac{{( {z}_{k} - m1 )}^{2}}{2 v1} - \frac{{( {z}_{k} - m2 )}^{2}}{2 v2} )$$

to exceed four standard deviations. Such a bounded range for the above quantity may be necessary if the expression data in Z exhibits a high level of noise or otherwise is not believed to be representative of the biological response represented in the data. When four standard deviations are selected as a floor, this represents a p-Value of less than 0.001, which allows for more accurate estimation of the normal distributions of the data points, particularly at the tails.

The probability that a sample Z is a member of group 1 may be computed as follows. Let mₖ₁, vₖ₁, mₖ₂, vₖ₂ be the mean and variances associated with the n selected marker genes Xₖ for groups 1 and 2, k=1 . . . n. An estimate of the probability that a sample Z, which includes expression levels for the n marker genes, is a member of group 1 is

$$P ( Z ∈ Group\_ 1 ) \approx ( \frac{1}{n} ) {\sum }_{k = 1 … n}^{n} {f}_{1} ( {z}_{k} , {m}_{k1} , {v}_{k1} , {m}_{k2} , {k}_{k2} )$$

Generally, a probability value for P above about 0.6 indicates that sample Z is a member of group 1, while a probability value for P below about 0.4 indicates that this sample is not a member of group 1. At values between 0.4 and 0.6, membership in or exclusion from group 1 cannot be determined. If these limits are changed from 0.6 and 0.4 to about 0.9 and 0. 1, there is very little effect on the model, because very few samples have a value for P in the range of 0.1 to 0.9.

Classification Database

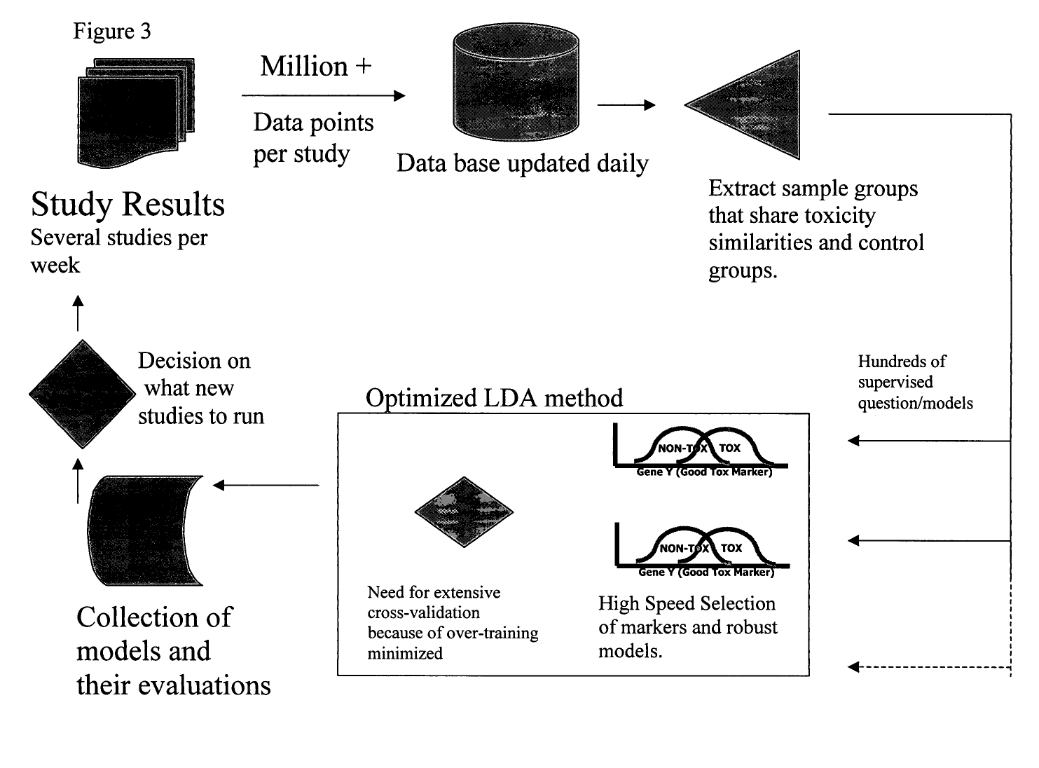

FIG. 3 illustrates a preferred methodology for building a fully informative classification database including the use of classification models for interpreting and classifying the results from biological studies contained in the database and for identifying areas where new studies are needed. In this aspect of the invention, information about biological processes is gathered and refined by a combination of meta-data, LDA modeling and follow-up biological studies.

The process of gathering and refining information in the database on a continual basis may include some or all of the following steps: performing studies; storing the results of these studies and the meta-data associated with the studies as samples in the database according to a supervised, unsupervised or combination supervised and unsupervised grouping procedure; selecting test and control groups for classification models; constructing and cross-validating markers derived from the models; evaluating the markers for their predictive abilities; and deciding where additional studies are needed for the database, including analysis of the models and/or meta-data. In a supervised grouping procedure, it is known from the published literature whether a sample belongs in the test group or in the control group. In an unsupervised grouping procedure, however, samples are placed in groups based only on measured data. During this process, the markers derived from the classification models are often used as a tool to assist in the decision of where additional studies are needed and, then, as these studies are performed, the resulting new samples are used to refine the markers. As more iterations of this process are performed, the more accurate the model becomes and, accordingly, more informed decision making can be for additional studies.

The following example refers to a classification database that uses gene expression as data points, but it is understood that other response types, e.g., protein expression, increase in production of metabolites or reaction intermediates, or any mass biological or chemical measure, may alternatively be used.

Biological studies are designed to capture events, such as toxicity, by exposing multiple animals or cell cultures to a stimulus such as a toxicant at multiple doses and negative controls. The information collected from these studies are stored in the database as samples, which may correspond to both gene expression data and meta-data about the study. The studies run over negative controls should cover a wide range of controls, such as negative controls, pharmacologic doses and vehicles. Which studies to run are initially determined by perceived need based on historical literature and dose ranging experiments where classical parameters (e.g., ALT/AST, BUN, histopathology scores, organ weight) are measured.

Several studies per week may be needed to meet the needs for research, and each of these studies may often exceed over a million data points per sample after measurement of gene expression levels. Meta-data may also be collected during each study. This meta-data can include study characteristics, histopathology, clinical chemistry, hematology and other assays. The meta-data may also supplement the gene expression data extracted from the studies to provide a broader picture of organ toxicity.

For example, one could query a toxicity expression database to learn what gene expression events preceded high ALT/AST ratios, which are a measure of cell lysis and the release of these enzymes into the blood. Because these enzymes are primarily found in liver cells, elevated ALT/AST ratios are an indication of liver damage and toxicity. One could also query a toxicity expression database to ask which genes are most associated with a particular type of histopathology, such as cholestasis, hepatitis, necrosis or peroxisomal proliferation.

After running the studies, the resulting samples are organized into tentative groupings in the database according to a specific protocol, such as a supervised grouping protocol. There are many supervised ways in which the samples may be reviewed to determine their appropriate grouping. When studies are initially run, the groupings may be determined by comparing samples across studies to look for common gene regulation which can best describe or discriminate perceived groupings. Both these and other types of perceived relationships (as may be assisted by the meta-data) can help to assemble tentative groups. But, it is assumed, as the name implies, that “tentative' groupings” will eventually turn out to be distinct from each other based on their respective distribution of data points, which represent gene expression patterns. Thus, a sample could be catalogued as a member of a particular group based on its perceived similarity in gene expression or the meta-data, but will later be catalogued under a different group when more studies are collected and more information is uncovered about the two related groups. Hundreds of supervised groups are contemplated for the databases of the invention, e.g., a toxicology database, examples of which are disclosed in Applicants' U.S. application Ser. No. 09/917,800, filed Jul. 31, 2001; Ser. No. 10/060,087, filed Jan. 31, 2002; Ser. No. 10/152,319, filed May 22, 2002; Ser. No. 10/301,856, filed Nov. 22, 2002; Ser. No. 10/191,803, filed Jul. 10, 2002; and Ser. No. 10/338,044, filed Jan. 8, 2003, all of which are incorporated herein by reference in their entirety.

After supervised groups are organized, it is desirable to identify one or more descriptors for discriminating one supervised group from another. An ideal descriptor would be one that not only can be used to describe group characteristics, e.g., a particular pathology, but which can also be used to predict samples that share the same physiological characteristics of the group. For example, if genes that are markers of toxicity are found to be related to separate treatments (possibly in separate studies) that result in necrosis of the liver in animals, these genes are much more informative if they can also predict whether a new, unknown sample will exhibit the same pathology when the animal or cell culture from which the sample is derived is exposed to any one of the treatments. Hence, these genes would be both predictive and descriptive.

In the context of building a fully informative database, the quality of a classification method for finding predictive combinations of genes is judged both by how predictive the results are and how difficult it is to implement and interpret the method. While methods of cross-validation have been developed to determine predictive capabilities of gene expression, it has been found that the methods are often too difficult to interpret and implement when they are used to build a rapidly expanding database. Accordingly, it has been determined that if a method is limited to identifying genes based on their predictive ability, i.e., closer to a method of gene selection for accomplishing the arduous task of determining predictive ability, the better this method is in the context of a rapidly expanding dynamic database where hundreds of models may be built and/or revised on a periodic bases.

Referring again to FIG. 3, in the LDA modeling procedure step, the method according to the LDA model embodiment described above is preferred. Other embodiments of an LDA model may also be used, provided they meet the requirements of streamlined, efficient cross-validation and marker selection and straightforward interpretation of results. By “straightforward,” it is meant that the properties of a particular set of markers are based essentially on the predictive ability of the individual markers. This stands in contrast to models in which a candidate marker's predictive ability, at least in part, is weighted by a cross-correlation, covariance or other assumption in the model that makes the analysis of a marker's predictive ability far more difficult, because its effect on the model is dependant on the measured responses of other markers.

After formulation and cross-validation of the LDA model, the markers and an interpretation of the predictive ability of the markers are stored in the database. One use of the models may be to predict a physiological change represented in samples that are not in the database. That is, the models may be used to predict biological classifications for externally submitted samples, such as samples supplied by customers. In this sense, a library of classification models is available as a service for biologists who wish to validate or better understand the biological process assumed to be represented in their samples.

Additionally, it may be useful to test the validity of an internally validated model, which is a model that is based upon samples derived from in-house studies, by evaluating the model's ability to predict the properties or toxic response of a sample submitted from an outside source. The better the external and internal results agree, the greater the confidence is that the internally generated model is properly validated. Additionally, more reliance can be placed on an initial description of the biological processes involved, for such purposes conducting future studies or interpreting the data collected. This is important because there is typically much more internally generated information for model evaluation than exists in externally submitted data. Nevertheless, external samples have two functions. They are valuable in validating the models of toxicity prediction, but once validated, the models may be used to evaluate the quality of external data sets, providing a means of quality control. An external data set that is at least of comparable quality to the internal database may be used alone or added to another database to predict toxicity for a group of samples derived from toxicity or toxicology studies.

LDA models may be effectively used to guide decisions on the content of new studies. In particular, LDA models may be used to assist with decisions on where new studies are needed to meet actual, as opposed to theoretical or perceived needs. Specifically, LDA models can assist with uncovering actual needs for biological data by a more or less brute force initiation of studies designed to isolate causes of a biological process by identifying areas where multiple studies are needed in order arrive at a training set that can then be used to uncover the markers that accurately discriminate between interesting and uninteresting physiological changes.

As would be expected, the more confidence there is in a model's ability to accurately reflect a true predictive ability, the better strategic decisions can be made on which new studies to run. But this predictive ability of the model must also be accompanied by an ability to quickly and efficiently update, cross-validate and interpret model results. This is important because the objectives of building an optimally informative database according to the invention, i.e., a database in which samples are collected for the purpose of fully informing the classification method of all possible causes for a particular biological process, requires that the model can be efficiently updated in response to new data, as well as easily interpretable when deciding on where new studies are needed. Accordingly, the value of a classification method in developing a fully informative classification database is measured by the extent to which data can be collected rapidly and in an organized manner. The amount of data made available to researchers depends upon the speed in which multiple studies can be run and models can be updated in response to this new data. Thus, reducing or eliminating the need for extensive cross-validation or the need for extensive external data for validation allows for rapid adjustment to study initiation and design of descriptive models. This saves valuable time and money by focusing on actual needs instead of relying on purely perceived, or thereotical needs for data generation derived from meta-data and/or complicated classification models that attempt, either directly or indirectly, to both explain and predict biological activity.

As mentioned earlier, an LDA model is well suited for re-evaluation of a perceived need because it weighs only the predictive ability of genes, and the set of genes designated as markers of toxicity can be efficiently updated in response to changes in the training set. This aspect of an LDA model in connection with building the database may be further understood by reference to the following example. Suppose that a general hepatoxicity LDA model exhibits good predictive ability for compounds A and B, but poor predictive ability for compound C. This may raise the question of whether or not there is an operative control in the studies for compound C, B, or A, or perhaps only C that affected the choice of markers for the model.

Such a situation may be illustrated by reference to FIG. 2. A general hepatotoxicity model provides more or less the same true-positive rates for predicting the toxicity of two of three hepatitis-inducing compounds (“HEP-A” and “HEP-B”, fourth and third pairs of bars, respectively) when these compounds are separately dropped from, or included in the training set for the LDA model, but the true-positive rate for predictions using the third compound (“HEP-C”, second pair of bars) drops by approximately 30% when the third compound is dropped from the training set. From this result, one may be able to derive only a more or less theoretical explanation for why HEP-C toxicity was not predicted by the model. For example, it may be concluded that there were improper controls or an interfering factor in one or more of the compound studies, perhaps even a confounding factor in studies using other compounds that was not present in the studies for HEP-C, but which affected the selection of markers for the model. Whatever the true cause was, the actual need for samples that will enable the identification of markers which can correctly predict toxicity upon treatment with HEP-C is most efficiently arrived at by running more studies. The selection of these studies is, however, an iterative process requiring perhaps several derivations of markers that will eventually fully inform the researcher of the true cause for the drop in the true positive rate for HEP-C.

One can also examine the compounds that do not predict well when dropped from the training set. Either similar compounds or ones that cause similar effects on meta-data parameters can then be added to the database. No one compound should be needed in the training set for it to predict correctly. The fully informative bioinformatic database is one that contains enough mutual information across compounds that true inference can be drawn. These markers should predict the presence of an effect in common among compounds, such as necrosis, rather than simply the presence of that compound. Because this type of LDA method is robust and easy to implement and interpret, a study drop cross-validation can be performed as compounds are added, and fast feed back as to what types of studies need to be performed can be quickly implemented.

In a preferred embodiment, the method for building a fully-informative database has been used to build a toxicology database which includes a library of LDA models that are used as predictive tools for various categories of toxicants. For example, the classifications and corresponding markers may be organized into one or more of the following types:

1. Hepatoxicants group vs. control group;

2. Pathology group including Necrosis, Steatosis, Cholestasis and Hepatitis vs. respective control groups for each pathology; and

3. Individual compound group vs. control group.

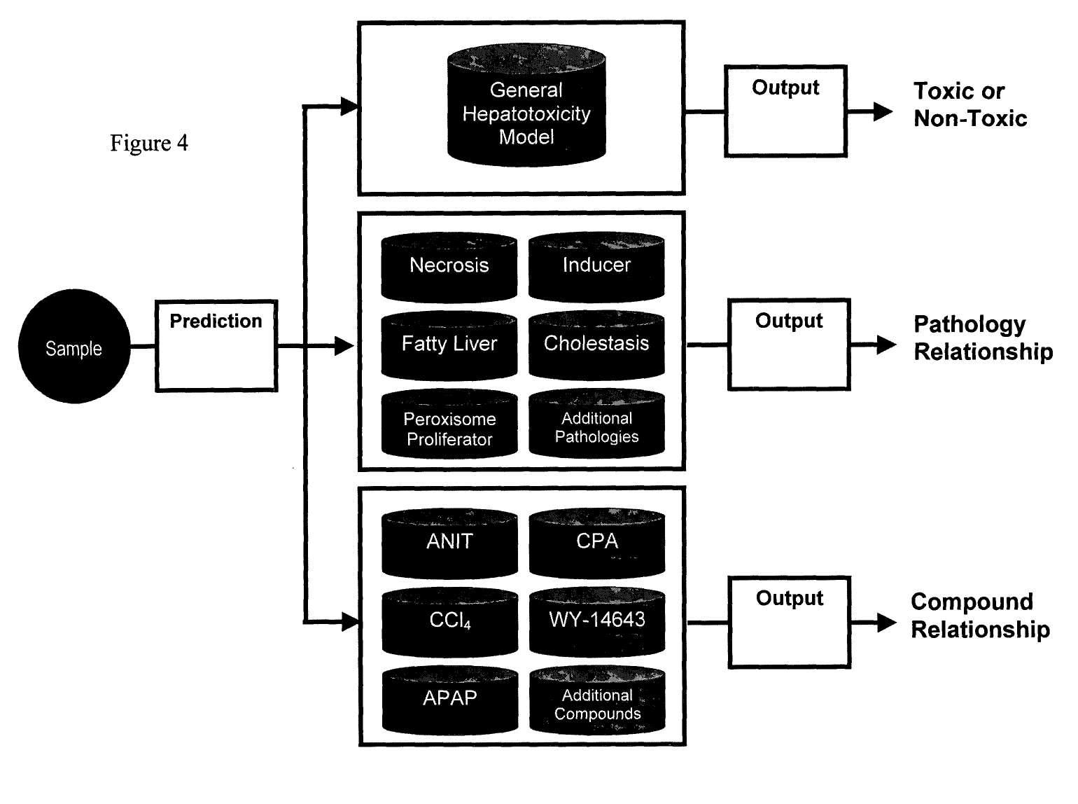

FIG. 4 illustrates the above classifications. In the models associated with these classifications, the control groups refer to any group of samples that are not the test group. For example, the control group for an individual compound would be all samples that are not taken from biological studies that include the target compound, the control group for the hepatitis group would be all samples that are not taken from a study on hepatitis, etc. When using the library of LDA models, one test group may be distinguished from another test group if desired, e.g., study samples predicted to develop hepatitis vs. study samples predicted to develop steatosis. A particular LDA model may be derived specifically for a distinguishing between two test groups. It is preferred, however, to maintain only one model for a particular classification (prediction of one disease). Thus, the same LDA model for predicting hepatitis is preferably used to determine which samples are likely to develop hepatitis as opposed to necrosis or steatosis.

Methods of Collecting Gene Expression Information

Any method may be used to produce gene expression data that may be used in and analyzed by the methods of the invention. For example, any nucleic acid detection assay format, including solution-based and solid support-based assay formats may be used. As used herein, “hybridization assay format(s)” refer to the organization of the oligonucleotide probes relative to the nucleic acid sample. The hybridization assay formats that may be used with the methods of the present invention include assays where the nucleic acid sample is labeled with one or more detectable labels, assays where the probes are labeled with one or more detectable labels, and assays where the sample or the probes are immobilized. Hybridization assay formats include but are not limited to: Northern blots, Southern blots, dot blots, solution-based assays, branched-DNA assays, PCR, RT-PCR, quantitative or semi-quantitative RT-PCR, microarrays and biochips.

As used herein, “nucleic acid hybridization” simply involves contacting a probe and nucleic acid sample under conditions where the probe and its complementary target can form stable hybrid duplexes through complementary base pairing (see Lockhart et al., WO 99/32660, published Jul. 1, 1999). The nucleic acids that do not form hybrid duplexes are then washed away leaving the hybridized nucleic acids to be detected, typically through detection of an attached detectable label.

It is generally recognized that nucleic acids are denatured by increasing the temperature or decreasing the salt concentration of the buffer containing the nucleic acids. Under low stringency conditions (e.g., low temperature and/or high salt) hybrid duplexes (e.g., DNA-DNA, RNA-RNA or RNA-DNA) will form even where the annealed sequences are not perfectly complementary. Thus, specificity of hybridization is reduced at lower stringency. Conversely, at higher stringency (e.g., higher temperature or lower salt) successful hybridization requires fewer mismatches. One of skill in the art will appreciate that hybridization conditions may be selected to provide any degree of stringency. In a preferred embodiment, hybridization is performed at low stringency, in this case in 6×SSPE-T at 37° C. (0.005% Triton x-100) to ensure hybridization, and then subsequent washes are performed at higher stringency (e.g., 1×SSPE-T at 37° C.) to eliminate mismatched hybrid duplexes. Successive washes may be performed at increasingly higher stringency (e.g., down to as low as 0.25×SSPET at 37° C. to 50° C. until a desired level of hybridization specificity is obtained. Stringency can also be increased by addition of agents such as formamide. Hybridization specificity may be evaluated by comparison of hybridization to the test probes with hybridization to the various controls that can be present (e.g., expression level control, normalization control, mismatch controls, etc.).

As used herein, the term “stringent conditions” refers to conditions under which a probe will hybridize to a complementary control nucleic acid, but with only insubstantial hybridization to other sequences. Stringent conditions are sequence-dependent and will be different under different circumstances. Longer sequences hybridize specifically at higher temperatures. Generally, stringent conditions are selected to be about 5° C. lower than the thermal melting point (Tm) for the specific sequence at a defined ionic strength and pH.

Typically, stringent conditions will be those in which the salt concentration is at least about 0.01 to 1.0 M sodium ion concentration (or other salts) at pH 7.0 to 8.3 and the temperature is at least about 30° C. for short probes (e.g., 10 to 50 nucleotides). Stringent conditions may also be achieved with the addition of destabilizing agents such as formamide.

In general, there is a tradeoff between hybridization specificity (stringency) and signal intensity. Thus, in a preferred embodiment, the wash is performed at the highest stringency that produces consistent results and that provides a signal intensity greater than approximately 10% of the background intensity. Thus, in a preferred embodiment, the hybridized array may be washed at successively higher stringency solutions and read between each wash. Analysis of the data sets thus produced will reveal a wash stringency above that the hybridization pattern is not appreciably altered and which provides adequate signal for the particular oligonucleotide probes of interest.

Probe arrays may contain at least two or more oligonucleotides that are complementary to or hybridize to one or more of the control genes described herein. Such arrays may also contain oligonucleotides that are complementary or hybridize to at least about 2, 3, 5, 7, 10, 50, 100 or more the genes described herein. Any solid surface to which oligonucleotides or nucleic acid sample can be bound, either directly or indirectly, either covalently or non-covalently, can be used. For example, solid supports for various hybridization assay formats can be filters, polyvinyl chloride dishes, silicon or glass based chips, etc. Glass-based solid supports, for example, are widely available, as well as associated hybridization protocols. (see, e.g., Beattie, WO 95/11755).

A preferred solid support is a high density array or DNA chip. This contains an oligonucleotide probe of a particular nucleotide sequence at a particular location on the array. Each particular location may contain more than one molecule of the probe, but each molecule within the particular location has an identical sequence. Such particular locations are termed features. There may be, for example, 2, 10, 100, 1000, 10,000, 100,000, 400,000, 1,000,000 or more such features on a single solid support. The solid support, or more specifically, the area wherein the probes are attached, may be on the order of a square centimeter.

1. Dot Blots

The methods of the present invention may be utilized in numerous hybridization formats such as dot blots, dipstick, branched DNA sandwich and ELISA assays. Dot blot hybridization assays provide a convenient and efficient method of rapidly analyzing nucleic acid samples in a sensitive manner. Dot blots are generally as sensitive as enzyme-linked immunoassays. Dot blot hybridization analyses are well known in the art and detailed methods of conducting and optimizing these assays are detailed in U.S. Pat. Nos. 6,130,042 and 6,129,828, and Tkatchenko et al. (2000), Biochimica et Biophysica Acta 1500:17-30. Specifically, a labeled or unlabeled nucleic acid sample is denatured, bound to a membrane (i.e., nitrocellulose) and then contacted with unlabeled or labeled oligonucleotide probes. Buffer and temperature conditions can be adjusted to vary the degree of identity between the oligonucleotide probes and nucleic acid sample necessary for hybridization.

Several modifications of the basic Dot blot hybridization format have been devised. For example, Reverse Dot blot analyses employ the same strategy as the Dot blot method, except that the oligonucleotide probes are bound to the membrane and the nucleic acid sample is applied and hybridized to the bound probes. Similarly, the Dot blot hybridization format can be modified to include formats where either the nucleic acid sample or the oligonucleotide probe is applied to microtiter plates, microbeads or other solid substrates.

2. Membrane-Based Formats

Although each membrane-based format is essentially a variation of the Dot blot hybridization format, several types of these formats are preferred. Specifically, the methods of the present invention may be used in Northern and Southern blot hybridization assays. Although the methods of the present invention are generally used in quantitative nucleic acid hybridization assays, these methods may be used in qualitative or semi-quantitative assays such as Southern blots, in order to facilitate comparison of blots. Southern blot hybridization, for example, involves cleavage of either genomic or cDNA with restriction endonucleases followed by separation of the resultant fragments on a polyacrylamide or agarose gel and transfer of the nucleic acid fragments to a membrane filter. Labeled oligonucleotide probes are then hybridized to the membrane-bound nucleic acid fragments. In addition, intact cDNA molecules may also be used, separated by electrophoresis, transferred to a membrane and analyzed by hybridization to labeled probes. Northern analyses, similarly, are conducted on nucleic acids, either intact or fragmented, that are bound to a membrane. The nucleic acids in Northern analyses, however, are generally RNA.

3. Arrays

Any microarray platform or technology may be used to produce gene expression data that may be used in the methods of the invention. Oligonucleotide probe arrays can be made and used according to any techniques known in the art (see for example, Lockhart et al., (1996), Nat Biotechnol 14:1675-1680; McGall et al. (1996), Proc Natl Acad Sci USA 93:13555-13460). Oligonucleotide probes used in the methods of the invention are preferably of sufficient length to specifically hybridize only to appropriate, complementary genes or transcripts. Typically the oligonucleotide probes will be at least about 10, 12, 14, 16, 18, 20 or 25 nucleotides in length. In some cases longer probes of at least 30, 40, or 50 nucleotides will be desirable. The oligonucleotide probes of high density array chips include oligonucleotides that range from about 5 to about 45 or 5 to about 500 nucleotides, more preferably from about 10 to about 40 nucleotides and most preferably from about 15 to about 40 nucleotides in length. In other particularly preferred embodiments, the probes are 20 or 25 nucleotides in length. In another preferred embodiment, probes are double- or single-stranded DNA sequences. The oligonucleotide probes are capable of specifically hybridizing to the control gene nucleic acids in a sample.

One of skill in the art will appreciate that an enormous number of array designs are suitable for the practice of this invention. The high density array will typically include a number of probes that specifically hybridize to each nucleic acid, e.g. mRNA or cRNA, in a sample (see WO 99/32660 for methods of producing probes for a given gene or genes). Assays and methods that can be used in the invention may simultaneously screen at least about 100, preferably about 1000, more preferably about 10,000 and most preferably about 500,000 or 1,000,000 different nucleic acid hybridizations.

A preferred oligonucleotide array may be selected from the Affymetrix, Inc. (Santa Clara, Calif.) GeneChip® series of arrays which include the Human Genome Focus Array, Human Genome U133 Set, Human Genome U95 Set, HuGeneFL Array, Human Cancer Array, HuSNP Mapping Array, GenFlex Tag Array, p53 Assay Array, CYP450 Assay Array, Rat Genome U34 Set, Rat Neurobiology U34 Array, Rat Toxicology U34 Array, Murine Genome U74v2 Set, Murine 11K Set, Yeast Genome S98 Array, E. coli Antisense Genome Array, E. coli Genome Array (Sense), Arabidopsis ATHI Genome Array, Arabidopsis Genome Array, Drosophila Genome Array, C. elegans Genome Array, P. aeruginosa Genome Array and B. subtilis Genome Array. In another embodiment, an oligonucleotide array may be selected from the Motorola Life Sciences and Amersham Pharmaceuticals CodeLink™ Bioarray System microarrays, including the UniSet Human 20K I, Uniset Human I, ADME-Rat, UniSet Rat I and UniSet Mouse I, or from the Motorola Life Sciences eSensor™ series of microarrays.

4. RT-PCR

The methods of the invention may be used in any type of polymerase chain reaction. A preferred PCR format is reverse transciptase polymerase chain reaction (RT-PCR), an in vitro method for enzymatically amplifying defined sequences of RNA (Rappolee et al. (1988), Science 241:708-712) permitting the analysis of different samples from as little as one cell in the same experiment (see “RT-PCR: The Basics,” Ambion, www.ambion.com/techlib/basics/rtpcr/index.html; PCR, M. J. McPherson and S. G. Møller, BIOS Scientific Publishers, Oxfordshire, England, 2000; and PCR Primer: A Laboratory Manual, Dieffenbach et al., Cold Spring Harbor Laboratory Press, Cold Spring Harbor, N.Y., 1995, for review). One of ordinary skill in the art may appreciate the enormous number of variations in RT-PCR platforms that are suitable for the practice of the invention, including complex variations aimed at increasing sensitivity such as semi-nested (Wasserman et al. (1999), Mol Diag 4:21-28), nested (Israeli et al. (1994), Cancer Res 54:6303-6310; Soeth et al. (1996), Int J Cancer 69:278-282), and even three-step nested (Funaki et al. (1997), Life Sci 60:643-652; Funaki et al. (1998), Brit J Cancer 77:1327-1332).

In one embodiment of the invention, separate enzymes are used for reverse transcription and PCR amplification. Two commonly used reverse transcriptases, for example, are avian myeloblastosis virus and Moloney murine leukaemia virus. For amplification, a number of thermostable DNA-dependent DNA polymerases are currently available, although they differ in processivity, fidelity, thermal stability and ability to read modified triphosphates such as deoxyuridine and deoxyinosine in the template strand (Adams et al. (1994), Bioorg Med Chem 2:659-667; Perler et al. (1996), Adv Prot Chem 48:377-435). The most commonly used enzyme, Taq DNA polymerase, has a 5′-3′ nuclease activity but lacks a 3′-5′ proofreading exonuclease activity. When fidelity is required, proofreading exonucleases such as Vent and Deep Vent (New England Biolabs) or Pfu (Stratagene) may be used (Cline et aL (1996), Nuc Acids Res 24:3456-3551). In another embodiment of the invention, a single enzyme approach may be used involving a DNA polymerase with intrinsic reverse transcriptase activity, such as Thermus thermophilus (Tth) polymerase (Bustin (2000), J Mol Endo 25:169-193). A skilled artisan may appreciate the variety of enzymes available for use in the present invention.

The methodologies and control gene primers of the present invention may be used, for example, in any kinetic RT-PCR methodology, including those that combine fluorescence techniques with instrumentation capable of combining amplification, detection and quantification (Orlando et al. (1998), Clin Chem Lab Med 36:255-269). The choice of instrumentation is particularly important in multiplex RT-PCR, wherein multiple primer sets are used to amplify multiple specific targets simultaneously. This requires simultaneous detection of multiple fluorescent dyes. Accurate quantitation while maintaining a broad dynamic range of sensitivity across mRNA levels is the focus of upcoming technologies, any of which are applicable for use in the present invention. Preferred instrumentation may be selected from the ABI Prism 7700 (Perkin-Elmer-Applied Biosystems), the Lightcycler (Roche Molecular Biochemicals) and iCycler Thermal Cycler. Featured aspects of these products include high-throughput capacities or unique photodetection devices.

Biological Samples

The biological samples used in the methods of the invention may be any tissue or cell sample taken from an animal, preferably a rat, a human, a dog or a mouse. Samples of the invention also include cell culture samples where a cell or cells were removed from an animal and grown in culture. Preferred tissue samples include those derived from liver, kidney, heart, brain or testicular tissue.

Toxins

The toxins used in studies to produce the predictive models described above include liver, kidney, heart and neurotoxins. The liver toxins include: acyclovir, amitryptiline, α-naphthylisothiocyanate (ANIT), APAP (acetominophen), CCl₄, AY-25329, bicalutamide, clofibrate, cyproterone acetate (CPA), diclofenac, diflunisal, dioxin, 1 7α-ethinylestradiol, hydrazine, indomethacin, bacterial lipopolysaccharide (LPS), phenobarbital, tacrine, valproate, WY-14643, zileuton, 2-acetylaminofluorene (2-AAF), BI liver toxin, chloroform, bicalutamide, CI-1000, colchicine, dimethylnitrosamine (DMN), dioxin, gemfibrozil, menadione, thioacetamide, lovastatin, methotrexate, tamoxifen, amiodarone, carbamazepine, chlorpromazine, imipramine and tetracycline.

Kidney toxins include: acyclovir, adriamycin, AY-25329, bromoethylamine HBr (BEA), carboplatin, cephaloridine, chloroform, cidofovir, cisplatin, citrinin, colchicine, cyclophosphamide, diclofenac, diflunisal, gentamicin, hydralazine, ifosfamide, indomethacin, lithium, menadione, mercuric chloride, pamidronate, puromycin aminonucleoside (PAN), sodium chromate, sodium oxalate, sulfadiazine, thioacetamide, and vancomycin.

Heart toxins include: cyclophosphamide, ifosfamide, minoxidil, hydralazine, BI-QT, clenbuterol, isoproterenol, norepinephrine, epinephrine, adriamycin, amphotericin B, epirubicin, phenylpropanolamine, and rosiglitazone, while an example of a neurotoxin used in the methods of the invention is 1-methyl-4-phenyl- 1,2,3,6-tetrahydropyridine HCl (MPTP).

Without further description, it is believed that one of ordinary skill in the art can, using the preceding description, practice the methods of the present invention. The examples given above are not to be construed as limiting the disclosure in any way. All references, patents and patent applications referred to in the application are incorporated by reference in their entirety.

What is claimed is:

1. A method of identifying one or more marker genes whose level of expression predicts a biological response in a cell or tissue, comprising:
(a) obtaining gene expression data for at least a test cell or tissue group and a control cell or tissue group, each of the test and control groups comprising a collection of cell or tissue samples;
(b) analyzing the gene expression data to identify said one or more marker genes by a linear discriminate metric that does not include a variable corresponding to a factor selected from the group consisting of: the magnitude of the difference in gene expression for each gene between the test and control groups, and the behavior or identity of other genes detected in the samples or across samples and combinations thereof, thereby identifying one or more marker genes whose level of expression predicts a biological response in a cell or tissue.

2. A method of claim 1, wherein the test cell or tissue group is exposed to a toxin.

3. A method of claim 1, wherein step (b) does not rely on a correlation matrix to account for relationships or interdependencies between the genes.

4. A method of claim 2, wherein step (b) comprises computing probability distributions from the gene expression data for each gene found in the toxin exposed cell or tissue group and the control cell or tissue group.

5. A method of claim 2, wherein the toxic response is hepatotoxicity.

6. A method of claim 2, wherein the toxin and control groups comprise at least about 10 samples.

7. A method of claim 2, wherein the toxin and control groups comprise at least about 50 samples.

8. A method of claim 2, wherein each gene is given equal weight as a predictive marker.

9. A method of claim 1, wherein step (b) comprises determining a scoring function and a discriminate score for each gene.

10. A method of claim 9, wherein the scoring function ƒ₁(Z) is generated according to the formula:
$${f}_{1} ( z ) = \frac{( \frac{1}{\sqrt{v1}} ) {ⅇ}^{- \frac{1}{2} {( {z}_{i} - m1 )}^{\frac{2}{v1}}}}{( ( \frac{1}{\sqrt{v1}} ) {ⅇ}^{- \frac{1}{2} {( {z}_{i} - m1 )}^{\frac{2}{v1}}} + ( \frac{1}{\sqrt{v2}} ) {ⅇ}^{- \frac{1}{2} {( {z}_{i} - m2 )}^{\frac{2}{v2}}} )}$$
wherein Z is a cell or tissue sample;
f₁(Z) is the discriminate score for sample Z relative to the samples of group 1;
m1 is an estimator of the true mean for the distribution of gene expression levels for the samples of group 1;
m2 is an estimator of the true mean for the distribution of gene expression levels for the samples of group 2;
v1 is an estimator of the variance in the distribution of gene expression levels for the samples of group 1; and
v2 is an estimator of the variance in the distribution of gene expression levels for the samples of group 2.

11. A method of claim 10, wherein a discriminate score for each gene is calculated by summing the instances where the scoring function returns a value for each sample that is greater than a predetermined significance level.

12. A method of claim 11, wherein the discriminate score (P) for each gene is calculated between the test and control groups by:
(a) counting the number of times, P1, that f1 (Xi)>½ for i=1 . . . t and the number of times, P2, that f1 (Yj)<½ for j=1 . . . n; and
(b) calculating the P discriminate score according to the formula: (P1+P2)/(n+t).

13. A method of claim 1, wherein the one or more marker genes discriminate between the test and control groups.

14. A method of claim 1, wherein the gene expression data comprises measurement of the amount or relative amount of mRNA for each gene.

15. A method of claim 1, wherein the one or more markers are cross-validated.

16. A method of claim 1, wherein the markers stratify a patient population.

17. A method of claim 16, wherein the patient population is stratified for its response to the administration of a drug or according to a physiological characteristic relevant to a clinical trial.

18. A method of building a classification database, comprising:
(a) obtaining gene expression data for at least one test group of cell or tissue samples and at least one control group of cell or tissue samples;
(b) identifying marker genes by an LDA metric capable of discriminating between the test and control groups and storing these markers in the database;
(c) evaluating the predictive ability of at least one of the markers of step (b); and
(d) refining the database with information derived from additional biological studies.

19. A method of claim 18, wherein the database comprises gene expression information and meta-data.
